(19) United States
(12) Patent Application Publication
(10) Pub. No.: US 2022/0196001 A1
(43) Pub. Date: Jun. 23, 2022

(54) PUMP DEVICE

(71) Applicants: Khaled ABOUSALEH, Schindellegi (CH); ZYNNON AG, Schindellegi (CH)

(72) Inventor: Khaled ABOUSALEH, Schindellegi (CH)

(21) Appl. No.: 17/526,500

(22) Filed: Nov. 15, 2021

(30) Foreign Application Priority Data
Dec. 21, 2020 (FR) 20 13783

(51) Int. Cl. F04B 13/00; F04B 17/03; F04B 49/06

(52) U.S. Cl. CPC F04B 13/00; F04B 49/06; F04B 17/03

(57) ABSTRACT

Disclosed is a precision pump using a syringe and of which a pusher for the piston of the syringe is equipped with a bending gauge.

### CROSS REFERENCE TO RELATED APPLICATION

This application is based on Patent Application FR 2013783 filed on Dec. 21, 2020, the entire contents of which are incorporated herein by reference in its entirety.

### BACKGROUND OF THE INVENTION

### Field of the Invention

The present invention relates to the field of syringe pumps, in particular precision pumps intended for dosing small volumes.

### Description of the Related Art

Precise dosing, in particular the case of drugs, can be vital. However, pumps may lose their precision in ageing. There may be leaks, for example at the syringe piston seals or at the connection of the syringe with a fluid inlet or outlet pipe. Clearances may appear or be aggravated between various components of the pump. Blockages or obstacles may also prevent normal operation of the pump.

The aim of the invention is to propose a pump equipped with means for monitoring the operation thereof, and to guarantee more precise dosing than in the pumps of the prior art.

### SUMMARY OF THE INVENTION

According to a first object of the invention, a system for actuating a piston of a syringe in a syringe pump, the piston being provided with a connector, comprises:
a motor;
transmission means; and
a pusher;
   the transmission means being designed to transmit an action of the motor to the pusher and the pusher being designed to move the piston in translation along a syringe axis,
   the pusher comprising:
a socket for the connector;
an attachment segment designed to be attached to the transmission means;
a beam that connects the socket to the attachment segment, transversely to the syringe axis; and
a sensor for measuring a bending of the beam about an axis perpendicular to the syringe axis.

The motor is preferably a rotary motor, a shaft of which forms a screw, the transmission means comprising a nut screwed onto the screw and a bracket translationally connected with this nut, the attachment segment of the pusher being attached to the bracket. The motor is advantageously a stepping motor.

The sensor may be a strain gauge glued to a face of the beam.

Advantageously, the socket and the connector form a linear-annular connection between each other. The connector may comprise a sphere formed at one end of an axial rod of the piston and the socket may comprise a cylindrical groove and a slot, the groove being designed for housing the sphere therein, extending along an axis substantially perpendicular to the syringe axis and having a diameter substantially equal to a diameter of the sphere, and the slot being designed for the passage of the rod with clearance when the sphere is in the groove. The groove may comprise an axial opening for introducing the connector therein.

According to a second object of the invention, a precision pump comprises a system according to the first object of the invention. It preferably comprises socket means for removably mounting therein a syringe according to the second object of the invention.

Such a pump advantageously comprises at least one sensor from a current sensor for the motor, a sensor for the pressure of the ambient air, an ambient temperature sensor, a vibration sensor, an infrared sensor, an ultrasound sensor and a moisture sensor. It may also comprise remote-control means and means for transmitting data read by the bending sensor and, where applicable, one or more other sensors at the remote-control means.

According to a third object of the invention, a method for controlling wear on a syringe in a pump according to the third object of the invention uses data read by the bending sensor and, where applicable, one or more other sensors. This method preferably comprises a prior learning step.

### BRIEF DESCRIPTION OF THE DRAWINGS

Several embodiments of the invention will be described below, by way of non-limitative examples, with reference to the accompanying drawings, in which:

FIG. 1 is a schematic view in three-quarter front left perspective, of a syringe pump according to the invention;

FIG. 2 is a schematic view in longitudinal section and in elevation of the pump of FIG. 1;

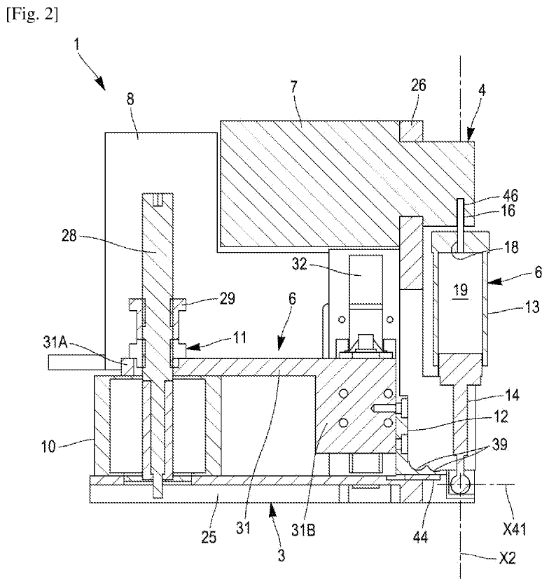

FIG. 3 is a schematic view in longitudinal elevation of the front part of the pump of FIG. 1, in the vicinity of the syringe;

FIG. 4 is a schematic view in three-quarter front right perspective, of a pusher for the syringe of FIG. 3;

FIG. 5 is a schematic elevation and front view of a detail of means for actuating the syringe; and

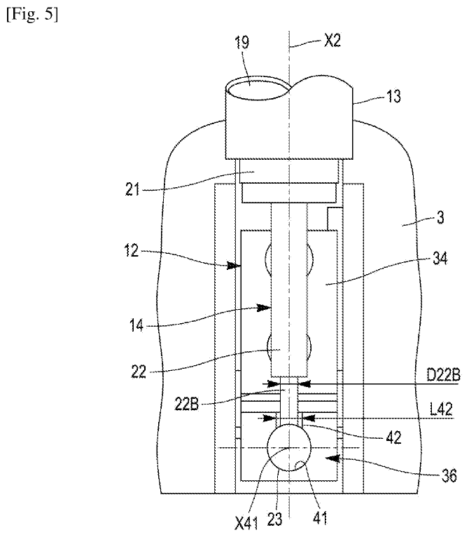

FIG. 6 is a schematic view in three-quarter front right perspective, similar to the one in FIG. 4, illustrating another embodiment of a pusher that can be used in the pump of FIG. 1.

### DESCRIPTION OF THE PREFERRED EMBODIMENTS

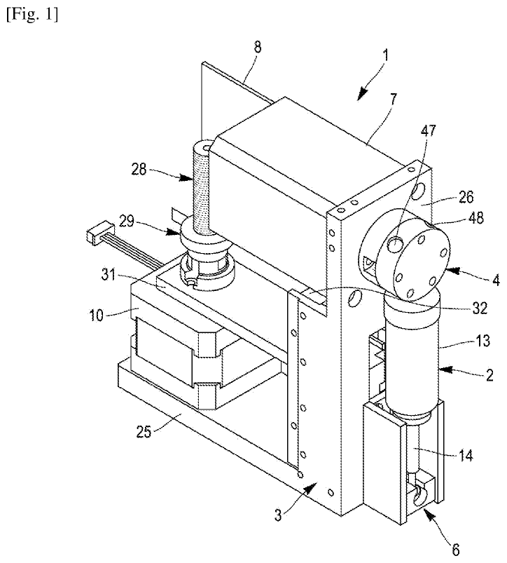

FIG. 1 shows a precision pump 1 according to the invention. This pump comprises and uses a syringe 2. It further comprises:
a chassis 3;
a valve 4;
means 6 for actuating the syringe;
a valve motor 7 for actuating the valve 4; and
electronic control means 8.
   The means 6 for actuating the syringe comprise a syringe motor 10, transmission means 11 and a pusher 12.

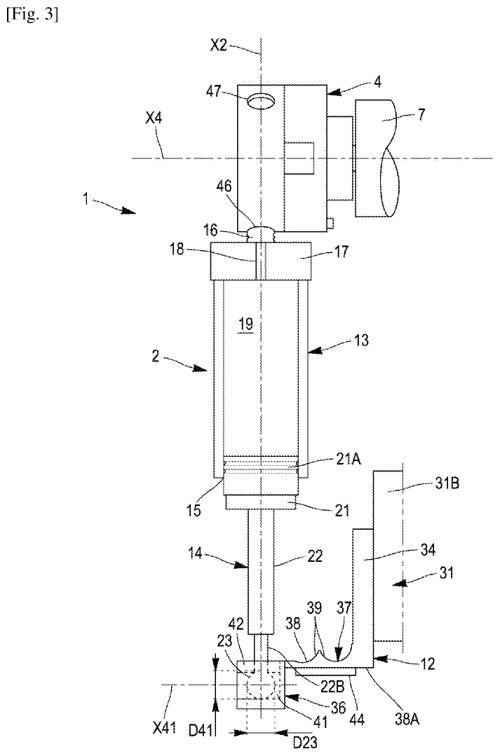

The syringe is substantially formed by rotation about a syringe axis X2. In the example illustrated, the syringe axis is vertical. As particularly illustrated in FIG. 3, the syringe 2 comprises a cylinder 13, a piston 14 able to move through an axial opening 15 in the cylinder, and an axial fluid socket 16. The fluid socket 16 is a male socket, formed outside the cylinder, opposite to the opening 15 in a bottom 17 of the cylinder. The syringe further comprises an axial channel 18 that allows a circulation of a fluid between a chamber 19 of the cylinder and the outside of the syringe, through the bottom 17 and the fluid socket 16. The chamber 19 is defined between the cylinder and the piston; the internal volume thereof varies according to the position of the piston in the cylinder.

In the example illustrated, the piston 14 comprises a head 21, mounted for sliding sealingly in the cylinder 13, a rod 22 that extends axially from the head to the outside of the cylinder, and a connector 23 formed at an end of the rod opposite to the head 21. The head comprises an annular seal 21A, for making a seal between the piston 14 and the cylinder 13. In the example illustrated, the connector 23 is in the form of a sphere 23 mounted at a part 22B with a reduced cross section of the rod 22. The rod and its part with a reduced cross section each have a circular cross section, around the syringe axis X2. The part with a reduced cross section has a constant diameter denoted D22B. The sphere 23 has a diameter denoted D23.

The valve 4 is a valve able to rotate about a valve axis X4. It is actuated by the valve motor 7. It comprises three ports 46-48. In the example illustrated, the three ports are female fluid sockets. A first female fluid socket 46 is designed to come into engagement with the male fluid socket 16 of the syringe. It further comprises a second female fluid socket 47 and a third female fluid socket 48, each for connecting a tube thereto. For example, a tube may be connected to a reserve in order to pump the fluid therein, and another tube for pouring a required volume of fluid thus pumped. A rotation of the valve makes it possible to make the first socket 46 communicate either with the second socket 47 or with the third socket 48.

The chassis 3 is produced by injection, from aluminum. It is in the form of an L. It comprises a base 25 in the form of a substantially rectangular plate elongated from front to rear. It also comprises a substantially vertical upright 26 that extends upwards from a front end of the base 25.

The motor 10 of the syringe is fixed to a rear end of the base 24. It comprises a shaft 28 disposed vertically and forming a screw. The transmission means 11 comprise a drive nut 29 and a bracket 31. The nut 29 is in engagement with the screw 28. The bracket extends from rear to front; it is in engagement by a rear end 31A with the nut 29 and in engagement with a linear guide 32 by a front end 31B. The guide 32 is attached to the upright 26 of the chassis 3. The nut drives the bracket 31 vertically and the guide 32 holds the bracket in vertical translation.

A linear encoder is attached to the chassis 3, and a graduated rule is attached to the bracket 31, facing the linear encoder. The movement of the graduated rule is read by a linear encoder. The linear encoder/rule pair makes it possible to check with great precision that a movement has taken place and makes it possible to determine a substantially absolute position of the piston relative to the chassis.

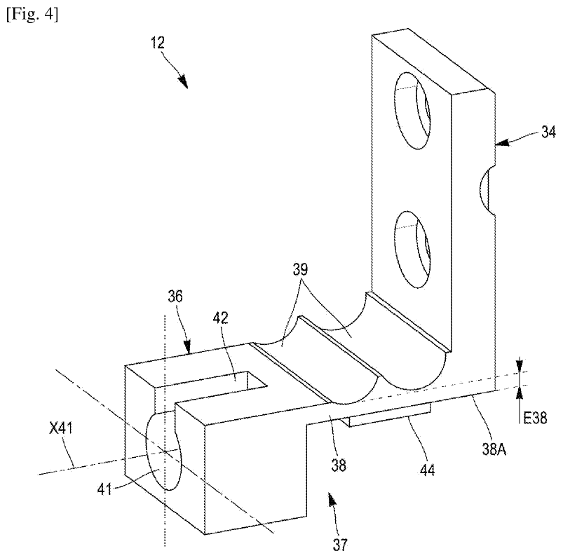

The pusher 12 is attached to the front end 31B of the bracket 31. As particularly illustrated in FIG. 4, the pusher 12 is in the form of an L, the upright of which is referenced 34 and the base of which is referenced 37. The upright 34 of the L serves as a segment for attaching the pusher to the bracket; it is pressed against a vertical front face of the front end 31B of the bracket 31; the upright 34 is attached thereto by screwing. The base 37 extends substantially horizontally, forwards, from a bottom end of the upright 34. The base 37 of the pusher comprises a socket 36 and a beam 38 that horizontally connects the socket to a bottom end of the upright 34 of the pusher 12.

The socket 36 of the pusher 12 comprises a cylindrical channel 41 around a substantially horizontal socket axis X41. The channel is open over the entire cross section thereof through a front face of the socket. It is also open in a top face of the socket, a slot connecting the channel with this top face. The channel 41 has a diameter D41 substantially equal to the diameter D23 of the sphere 23, so that the sphere forms a linear-annular connection with the channel and can slide therein, along the socket axis X41. The slot 42 has a width L42 greater than the diameter D22B of the reduced cross section 22B of the rod 22 of the syringe 6, so that a clearance is formed on either side of the part 22B of the rod, so as to allow a tilting of the rod about the socket axis X41.

The syringe 2 is demountable, i.e. it is possible to replace one syringe with another. To fit a syringe, it is necessary to put the male fluid socket 16 and the first female fluid socket 46 facing each other, to insert the connector 23, here the sphere 23, in the socket 36, so that the axis X2 of the syringe is substantially vertical, and then to screw together the fluid sockets 16, 46.

The pusher makes it possible, through the action of the channel on the sphere, to push upwards, and to pull downwards, the piston 14. The freedom of movement of the sphere 23 in the channel 41 guarantees that the action of the pusher on the rod is directed along the axis X2 of the syringe.

The beam 38 comprises a substantially horizontal bottom surface 38A. A strain gauge 44 is attached to the bottom face 38A. This gauge is designed to measure bending stresses about a horizontal axis transverse to the beam. The stresses thus measured are essentially dependent on the forces exerted by the syringe 2 on the pusher 12.

When the pusher 12 moves and moves with it the piston 14 of the syringe 2, forces are generated on the pusher. These forces result from the friction forces of the seal 21A on the piston and from the pressure of the fluid located in the chamber 19 of the syringe. The gauge 44 makes it possible in particular to evaluate the friction forces of the seal and the wear on the seal.

Weakening zones 39 are formed in the top part of the beam 38. They make it possible to increase the sensitivity of the gauge 44. They are disposed so as to keep sufficient stiffness for the beam 38 to guarantee satisfactory precision of the movement of the piston 14, and therefore of the dosing. In the example illustrated, the weakening zones consist of two semi-cylinders 39, transverse to the beam, open towards the top in a top face of the beam. A current thickness of the beam, measured between the bottom face and the bottom of the weakening zones 39, is denoted E38. It is this thickness that determines the range of forces that the gauge 44 is capable of measuring.

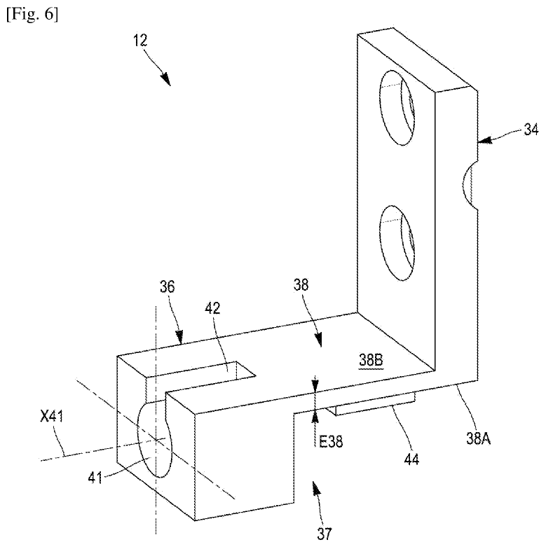

FIG. 6 illustrates a second embodiment for the pusher 12. It differs from the first embodiment, illustrated in FIG. 4, in that the cross section of the beam 38, transversely to the socket axis X41, is substantially constant and rectangular. In this configuration, if it makes it possible to have uniform stresses in the beam, for the same current thickness E8 of the beam, the bending movement of the beam is greater than the same movement in the case of the first embodiment.

In this second embodiment, the gauge 44 can be attached to a top face 38B of the beam 38, instead of being disposed on the bottom face 38A. Thus it is possible to use two gauges, one on the bottom face 38A, the other on the top face 38B.

Measuring the friction forces of the seal in real time makes it possible to monitor the change in the wear on the seal over time and makes it possible to prevent leaks and to anticipate the replacement of the seal, or the replacement of the syringe as a whole.

In the case where a system according to the invention is used for dosing, in the context of a diagnosis, the risks of errors are reduced, which is beneficial for the patient concerned.

Measurement of wear on the seal is very important since it makes it possible to avoid leaks and therefore dosing errors that result therefrom. When the pump is a medication pump connected to a perfusion, this reduces the risks for a patient thus perfused.

Measuring this wear also makes it possible to plan the replacement of the syringe and to do it at the correct time, that is to say neither too early nor too late. The replacement is thus based on a physical measurement.

Furthermore, the pump 1 comprises control means for each of the motors.

Any variation in pressure generated in the chamber is transformed into a force and is measured by the strain gauge.

With a single sensor, here the gauge 44, it is possible to measure both wear and pressure.

The motor 10 that actuates the pusher 12 and the motor 7 that actuates the valve 4 are stepping motors. The control means 8 comprise control means for the motors 7, 10 comprising, for each motor 7, 10, a current sensor. Each current sensor makes it possible to implement the following functions:
Controlling the heating of the motor, which results from the current circulating in the motor;
Controlling the torque of the motor, in particular of the syringe motor 10, since there is a link between current and torque;
Predicting the wear on the motor, a link existing between the current consumed and the wear on the pump. Remote monitoring is advantageously implemented. An application comprising an algorithm, based on artificial intelligence, can advantageously be provided for aiding the remote monitoring.

The pump also comprises a sensor that makes it possible to monitor the total electrical consumption thereof, which, apart from controlling the total consumption, makes it possible to predict a total of remaining service life for the pump 1.

The pump may advantageously comprise other sensors, in particular selected from a sensor for the pressure of the ambient air, an ambient temperature sensor, a vibration sensor, an infrared sensor, an ultrasound sensor and/or a moisture sensor.

There is a link between the pressure of the air and the dosing function of the pump. This is because the dosing function is a relationship between the variation in pressure generated by the movement of the piston and the atmospheric pressure present at the end of the tube (or sampling needle). Therefore the sensor for the pressure of the ambient air, i.e. the pressure of a place where the pump is located, makes it possible to correct the volume generated by the dosing function.

There is also a link between volume and temperature. By virtue of the ambient temperature sensor, the dosing function is corrected or adjusted according to the ambient temperature. This temperature sensor also makes it possible to ensure that the product is functioning in a specified temperature range.

The pump 1 comprises several mechanical movements that generate vibrations. The vibration sensor makes it possible to monitor the change in the vibration levels in order to predict and anticipate mechanical breakdowns as well as malfunctions.

The objective of the ultrasound sensor is to measure a sound generated by the mechanical movements, also in order to predict and/or anticipate mechanical breakdowns as well as malfunctions.

An infrared sensor is advantageously directed towards each of the motors 7, 10. It makes it possible to measure without contact the actual temperature of the motor. Since the pump 1 is generally installed in a confined environment, for example inside an analysis machine, a precise monitoring of the temperatures of the motors and of overheating in general is advantageous.

The moisture sensor makes it possible to measure the ambient moisture. It makes it possible to ensure that the pump is functioning in a specified moisture range. Furthermore, in the event of leakage of the pump, the moisture level increases and the moisture sensor can thus make it possible to detect such a leak.

The control means 8 of the pump 1 advantageously comprise means of connection to a remote control device and/or for example to the “cloud”. These connection means may comprise a cellular, Bluetooth and/or Wi-Fi connection. These connection means make it possible to send data or measurements collected by the sensors to the remote control means, in real or deferred time.

The control means 8 of the pump are advantageously designed to receive incoming information. This incoming information may be specific configurations or an update of a software.

Sending data from the sensors to the cloud in order to analyze them in real or offset time makes it possible to provide the user with important services such as:
a prediction of wear and anticipation of replacement of the syringe;
a prediction of a maintenance requirement;
a prediction of breakdown;
a prediction of end of life; and/or
a provision of alarms or information when the use of the product deviates from the specifications, for example from a specific range of temperatures or moisture, in order to guarantee that the dosing results are in accordance with the expectations of a user.

A syringe pump according to the invention has the following features:
the linear movement of the piston 14 is achieved by means of a mechanical connection between a screw 28 and a nut 29 whereas the pumps of the prior art use a belt. The screw/nut connection provides the following improvements:
   mechanical simplicity, which reduces the number of parts;
   reduction in the total mechanical clearances, which improves the precision of the pump;
   saving in space, because of a reduced number of parts;
   higher speeds and accelerations are possible;
   simplified manufacture since it does not require adjusting the tension of a belt; and
   longer service life than the service life of a belt.
the linear movement of the piston 14 is furthermore provided by the linear guide 32 in order to withstand the forces;
the function of the pusher 12 is to push and pull the piston 14 of the syringe 2 in order to fulfill the dosing function.

One and the same pump according to the invention can be designed to accept a plurality of sizes of syringe, for example syringes able to contain 100 μl, 250 μl, 500 μl, 1000 μl, 2.5 ml, 5 ml, 12.5 μl, 25 ml or 50 ml. Such a pump may be designed to dose with precision volumes lying between approximately 0.1 μl and 50 ml.

Naturally the invention is not limited to the examples that have just been described. On the contrary, the invention is defined by the claims that follow.

It will in fact be clear to a person skilled in the art that various modifications can be made to the embodiments described above, in the light of the teaching that has just been disclosed to him.

In particular, the syringe axis may not be vertical. The pump may also comprise a plurality of syringes, driven by the same pusher or not.

In addition, instead of being made from aluminum, the chassis may also be made from a plastics material.

1. System for actuating a pump of a syringe in a syringe pump, the piston being provided with a connector, comprising:
a motor;
transmission means; and
a pusher;
the transmission means being designed to transmit an action of the motor to the pusher and
the pusher being designed to move the piston in translation along a syringe axis,
the pusher comprising:
a socket for said connector;
an attachment segment designed to be attached to the transmission means;
a beam that connects the socket to the attachment segment, transversely to the syringe axis; and
a sensor for measuring a bending of the beam about an axis perpendicular to the syringe axis.

2. The system according to claim 1, wherein the motor is a rotary motor comprising a shaft forming a screw and wherein the transmission means comprise a nut screwed onto the screw and a bracket translationally connected with said nut, the attachment segment of the pusher being attached to said bracket.

3. The system according to claim 1, wherein the motor is a stepping motor.

4. The system according to claim 1, wherein the sensor is a strain gauge glued to a face of the beam.

5. The system according to claim 1, wherein the socket and the connector form a linear-annular connection between them.

6. The system according to claim 5, wherein the connector comprises a sphere formed at one end of an axial rod of the piston and wherein the socket comprises a cylindrical groove and a slot, said groove being designed to house said sphere therein, extending along an axis substantially perpendicular to the syringe axis and having a diameter substantially equal to a diameter of said sphere, and said slot being provided for the passage with clearance of said rod when the sphere is in said groove.

7. The system according to claim 6, wherein the groove comprises an axial opening for introducing the connector therein.

8. Precision pump, comprising a system according to claim 1.

9. The precision pump according to claim 8, comprising socket for removably mounting a syringe therein.

10. The precision pump according to claim 8, further comprising at least one sensor from a current sensor for the motor, a sensor for the pressure of the ambient air, an ambient temperature sensor, a vibration sensor, an infrared sensor, an ultrasound sensor and a moisture sensor.

11. The precision pump according to claim 8, further comprising remote-control means and means for transmitting data read by the bending sensor and, where applicable, one or more other sensors to said remote-control means.

12. Method for monitoring wear on a syringe in a pump according to claim 8, wherein the method uses data read by the bending sensor and, where applicable, one or more other sensors.

13. The method according to claim 12, further comprising a prior learning step.

14. The system according to claim 2, wherein the motor is a stepping motor.

15. The system according to claim 2, wherein the sensor is a strain gauge glued to a face of the beam.

16. The system according to claim 3, wherein the sensor is a strain gauge glued to a face of the beam.

17. The system according to claim 2, wherein the socket and the connector form a linear-annular connection between them.

18. The system according to claim 3, wherein the socket and the connector form a linear-annular connection between them.

19. The system according to claim 4, wherein the socket and the connector form a linear-annular connection between them.

20. Precision pump, comprising a system according to claim 2.
